(19) United States
(12) Patent Application Publication
(10) Pub. No.: US 2022/0370748 A1
(43) Pub. Date: Nov. 24, 2022

(54) PASSIVE OXYGEN MASK VACUUM REGULATION SYSTEM

(71) Applicant: POM Medical, LLC, Simi Valley, CA (US)

(72) Inventor: Edward Lehman, Las Vegas, NV (US)

(73) Assignee: POM Medical, LLC, Simi Valley, CA (US)

(21) Appl. No.: 17/470,911

(22) Filed: Sep. 9, 2021

Related U.S. Application Data
(60) Provisional application No. 63/079,317, filed on Sep. 16, 2020.

(51) Int. Cl. A61M 16/06; A61M 16/08

(52) U.S. Cl. CPC A61M 16/0672; A61M 16/085; A61M 16/0858; A61M 2202/0208; A61M 2205/3331

(57) ABSTRACT

A passive oxygen mask and vacuum regulation system disposed in fluid communication with a vacuum scavenging array includes a passive oxygen mask dimensioned to fit over a patient's nose and mouth and having a gas inlet port and an expired gas outlet port. A vacuum regulation assembly has a vacuum regulator which includes an expired gas discharge tube disposed in fluid communication between the passive oxygen mask and the vacuum regulation assembly, and a vacuum suction tube disposed in fluid communication between the vacuum regulation assembly and the vacuum scavenging array. The vacuum regulation assembly further comprising a vacuum attenuation port through a portion of a regulator housing, wherein the vacuum attenuation port is at least partially defined by an attenuation port diameter dimensioned to reduce a vacuum suction pressure in the expired gas discharge tube to a predetermined vacuum suction pressure.

### CROSS-REFERENCE TO RELATED APPLICATIONS

This application claims the benefit of U.S. Provisional Patent Application Ser. No. 63/079,317 filed on Sep. 16, 2020, which is incorporated by reference herein in its entirety.

### FIELD OF THE INVENTION

The present invention relates generally to a passive oxygen mask and vacuum regulation system.

### BACKGROUND OF THE INVENTION

A medical face mask is a commonly used medical device which facilitates introduction of oxygen and/or other medical gases or aerosols, such as, by way of example, anesthetics, antibiotics, steroids, etc., to a patient for inhalation into the patient's lungs. A conventional medical face mask may be configured to engage the face and cover the nose and mouth of the patient in an airtight or non-airtight manner. Medical face masks are typically fabricated of a resilient material such as plastic, silicone or rubber and are typically disposable. Medical face masks may be transparent to enable healthcare providers to assess the patient, as well as to reduce the patient's perception of claustrophobia.

Medical face masks are utilized to facilitate accuracy in the quantities or doses of oxygen and/or anesthetic or other medical gases which are administered to a patient in various medical applications. In general, medical face masks are generally classified as either a “positive ventilation” type of mask or a passive “non-positive ventilation” type of mask.

It is understood that excess or insufficient quantities of oxygen or other medical gases are potentially harmful to a patient's health. Because of this, the quantities of oxygen and medical gases and oxygen/gas mixtures which are administered to a patient are closely monitored. Thus, the design of a medical face mask is important to assure the delivery of oxygen and medical gases in accurate amounts or dosages to the patient.

A “positive ventilation” medical face mask is configured such that a positive flow of oxygen and/or other medical gases are constantly supplied to the mask under a positive pressure. Of course, it then becomes necessary to capture excess oxygen and/or other medical gases which a patient does not inhale from such a “positive ventilation” medical face mask, so as to prevent excess oxygen and/or other medical gases from building up in the room in which the patient is receiving oxygen and/or other medical gases via a positive ventilation medical face masks mask, which can present a fire or explosive hazard, as well as potentially exposing medical personal to anesthetics or other medical gases intended for the patient.

As such, modern hospitals have a central vacuum scavenging system which includes an interface in patient rooms, typically behind the head of a patient bed, as well as in operating theaters, examination rooms, etc. These vacuum scavenging systems include a vacuum intake to which an evacuation tube may be interconnected to a “positive ventilation” medical face mask to constantly draw off excess oxygen and/or other medical gases from the medical face mask. These central vacuum scavenging systems typically operate at an elevated vacuum suction pressure of about 100 to about 700 millimeters of mercury vacuum. As will be appreciated, these elevated amounts of vacuum suction pressure will adversely affect operation of the “positive ventilation” medical face mask because the hospital central vacuum scavenging system suction pressure can be greater than the positive pressure of the supplied oxygen, gas, or anesthetics which could result in dangerous life threatening mixtures and/or death.

A passive or “non-positive ventilation” medical face mask is configured such that oxygen and/or other medical gases are supplied on an on demand basis to the mask, and thus, to the patient, however, unlike a “positive ventilation” medical face mask, oxygen and/or other medical gases are not supplied to a “non-positive ventilation” medical face mask under pressure. However, similar to a “positive ventilation” medical face mask, it is often still necessary to capture excess oxygen and/or other medical gases exhaled by a patient into such a “non-positive ventilation” medical face mask, so as to prevent oxygen and/or other medical gases from escaping into the room in which the patient is present. Moreover, and especially under current conditions wherein containment of the airborne virus which causes the novel coronavirus known as COVID-19, it is critical, and perhaps life-saving, to capture the expired gases exhaled by a patient into a passive or “non-positive ventilation” medical face mask.

It is appreciated that it is possible to utilize a central vacuum scavenging system via an interface in a patient room having a vacuum intake interconnected to an evacuation tube from a “non-positive ventilation” medical face mask to constantly draw off excess oxygen, other medical gases, and/or airborne viruses or other pathogens exhaled by a patient into such a “non-positive ventilation” medical face mask. However, it is further appreciated that a vacuum suction pressure of 100 millimeters of mercury, i.e., 100 millimeters of mercury negative gauge pressure, would essentially draw oxygen and/or other medical gases supplied to a “non-positive ventilation” medical face mask out from the mask before a patient has an opportunity to inhale and process the oxygen or other medical gases, thereby defeating the purpose of a “non-positive ventilation” medical face mask itself. Furthermore, a vacuum suction pressure of as little as 100 millimeters of mercury negative gauge pressure would significantly interfere with the sampling and measurement of vital respiration parameters of a patient wearing such a “non-positive ventilation” medical face mask, such as, by way of example, an end tidal carbon dioxide (ETCO2), or a fraction of inspired oxygen (FiO2). However, once again, it is often desirable, and in some cases it is mandated, to capture the expired gases exhaled by a patient wearing a “non-positive ventilation” medical face mask. This becomes particularly important when treating patients infected with airborne viruses and/or other airborne pathogens, such as, by way of example, a patient infected with the novel coronavirus known as COVID-19.

Accordingly, there is an established need for a solution to operation of a passive “non-positive ventilation” medical face mask with standard central vacuum scavenging systems found in modern hospitals today.

### SUMMARY OF THE INVENTION

The present invention is directed to passive oxygen mask and vacuum regulation system disposed in fluid communication with a vacuum scavenging array via a vacuum interface.

In a first implementation of the invention, a passive oxygen mask and vacuum regulation system disposed in fluid communication with a vacuum scavenging array via a vacuum interface comprises: a passive oxygen mask defining an internal volume configured and dimensioned to fit over a patient's nose and mouth, the mask having a gas inlet port and an expired gas outlet port; a vacuum regulation assembly comprising a vacuum regulator having a regulator housing which includes a mask interconnect and a vacuum array interconnect; the vacuum regulation assembly further comprising an expired gas discharge tube disposed in fluid communication between the passive oxygen mask and the vacuum regulation assembly; the vacuum regulation assembly also including a vacuum suction tube disposed in fluid communication between the vacuum regulation assembly and the vacuum scavenging array; and the vacuum regulation assembly further comprising a vacuum attenuation port through a portion of the regulator housing, wherein the vacuum attenuation port is at least partially defined by an attenuation port diameter dimensioned to reduce a vacuum suction pressure in the expired gas discharge tube to a predetermined vacuum suction pressure.

In a second aspect, the passive oxygen mask and vacuum regulation system can include a passive oxygen mask further comprising a sampling port disposed in fluid communication with an internal volume thereof to permit sampling of a gaseous mixture from the internal volume of the passive oxygen mask for analysis.

In another aspect, the passive oxygen mask and vacuum regulation system may have a passive oxygen mask further comprising a gas reservoir disposed in fluid communication with an internal volume thereof.

In a further aspect, the passive oxygen mask and vacuum regulation system can include a passive oxygen mask having a nasal instrument insertion port.

In one other aspect, the passive oxygen mask and vacuum regulation system may have a passive oxygen mask including an oral instrument insertion port.

In a further aspect the passive oxygen mask and vacuum regulation system can have a passive oxygen mask including a tacky or clingy silicon polymer affixed or adhered onto a mask flange to strengthen the seal between the mask flange and the surface of the patient's face.

In still another aspect, the passive oxygen mask and vacuum regulation system may have a vacuum regulation assembly including a fixed vacuum attenuation port through a portion of a regulator housing, the fixed vacuum attenuation port at least partially defined by a fixed attenuation port diameter dimensioned to reduce a vacuum suction pressure in an expired gas discharge tube to a predetermined vacuum suction pressure, wherein the predetermined vacuum pressure is in a range of about 75 millimeters of mercury negative gauge pressure to about 25 millimeters of mercury negative gauge pressure.

In yet a further aspect, the passive oxygen mask and vacuum regulation system can include a vacuum regulation assembly further comprising a variable vacuum attenuation port through a portion of a regulator housing, the variable vacuum attenuation port at least partially defined by a variable attenuation port diameter dimensioned to reduce a vacuum suction pressure in an expired gas discharge tube to one of a plurality of predetermined vacuum pressures, wherein the plurality of predetermined vacuum pressures are in a range of about 75 millimeters of mercury negative gauge pressure to about 25 millimeters of mercury negative gauge pressure.

In still one other aspect, the passive oxygen mask and vacuum regulation system may have a vacuum regulation assembly including a vacuum sensor disposed in fluid communication with an expired gas discharge line so as to allow measurement of a vacuum pressure therein.

In yet another aspect, the passive oxygen mask and vacuum regulation system can include a vacuum regulation assembly having a variable attenuation port controller disposed in communication with at least a vacuum sensor disposed in an expired gas discharge tube.

In still a further aspect, the passive oxygen mask and vacuum regulation system may have a vacuum regulation assembly including a variable attenuation port actuator operative with a variable vacuum attenuation port so as to vary a variable attenuation port diameter thereof.

In yet one other aspect, the passive oxygen mask and vacuum regulation system can include a variable attenuation port controller disposed in communication with a variable attenuation port actuator, the variable attenuation port controller configured to direct the variable attenuation port actuator to expand or contract the variable attenuation port diameter so as to vary a vacuum pressure in an expired gas discharge tube as detected by a vacuum sensor to one of one of plurality of predetermined vacuum pressures.

In still another embodiment, the passive oxygen mask and vacuum regulation system is configured to direct expired patient gasses into an expired gas outlet port and through a series of filters, lights, sounds, baffles, electromagnetic wavelengths, ultraviolet (UV) light, radio waves, which are not limited to a particular spectrum of light or energy, located in an expired gas discharge tube.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a front elevation of one illustrative embodiment of a passive oxygen mask and vacuum regulation system, in accordance with the present invention;

FIG. 2 presents a partial front perspective view of the passive oxygen mask and vacuum regulation system of FIG. 1, in accordance with the present invention;

FIG. 3 presents a front elevation of one alternative illustrative embodiment of a passive oxygen mask and vacuum regulation system, in accordance with the present invention; and

FIG. 4 presents a partial front elevation of the alternative embodiment of a passive oxygen mask and vacuum regulation system of FIG. 3, in accordance with the present invention.

Like reference numerals refer to like parts throughout the several views of the drawings.

### DETAILED DESCRIPTION

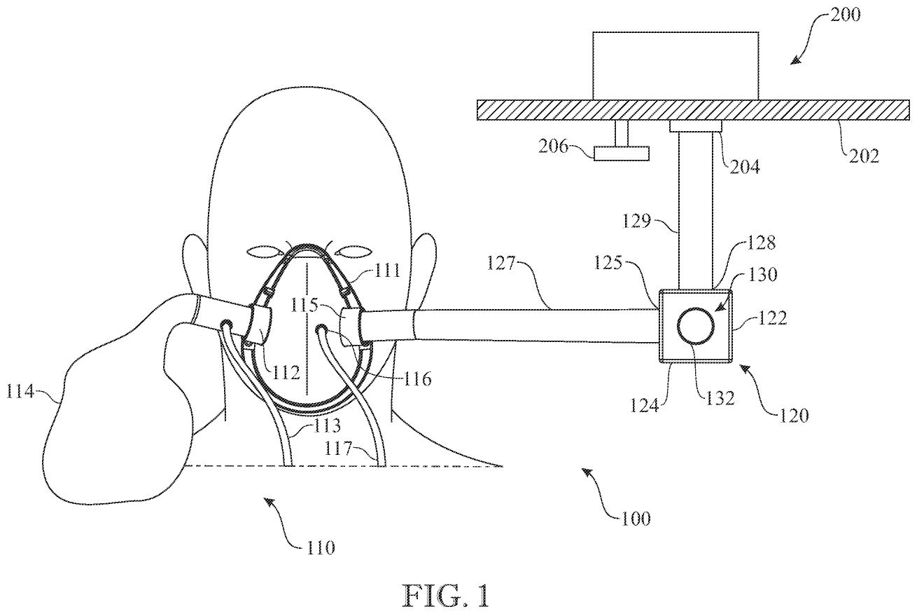

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “top”, “bottom”, “left”, “right”, “front”, “rear”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed to a passive oxygen mask and vacuum regulation system disposed in fluid communication with a vacuum scavenging array.

Referring initially to FIG. 1, presented therein is a front elevation of one illustrative embodiment of a passive oxygen mask and vacuum regulation system in accordance with the present invention, generally as shown as at 100 throughout the figures. As may be seen from FIG. 1, a passive oxygen mask and vacuum regulation system 100 comprises a passive oxygen mask 110, also referred to as a “non-positive ventilation” oxygen mask, such as are utilized to provide oxygen and/or other medical gases, such as, by way of example only, vaporized or aerosolized medications and/or anesthetics, in an “on-demand” manner as a patient wearing the passive oxygen mask inhales. Thus, operation of a passive oxygen mask 110 in accordance with the present system 100 is completely different to that of a positive pressure or “positive ventilation” oxygen mask where a constant flow of oxygen and/or other medical gases is constantly provided to the patient via such a positive pressure oxygen mask. More in particular, the present system 100 permits the effective vacuum suction pressure of a central hospital vacuum scavenging system, which is vital to capture the contaminated expired gases escaping the “positive ventilation” medical face mask, to be controlled and/or attenuated without compromising the administration of intended gasses and/or anesthetics.

With continued reference to FIG. 1, a passive oxygen mask 110 in accordance with at least one embodiment the present invention comprises a gas inlet port 112 to facilitate interconnection to a supply of oxygen and/or other medical gases into an interior of the passive oxygen mask 110, such as, via a gas supply line 113, so that the oxygen and/or other medical gas is always readily available for inhalation by a patient wearing the passive oxygen mask 110. A mask flange 111 is provided around the periphery of a positive oxygen mask 110 in accordance with the present invention so as to provide a comfortable yet positive seal around a patient's nose and mouth, such as is shown in the illustrative embodiment of FIG. 1.

In at least one embodiment, a tacky or clingy silicone or other plastic polymer is affixed or adhered onto a mask flange 111 of a passive oxygen mask 110 to strengthen the positive seal between the mask flange 111 and the surface of the patient's face. More in particular, the tacky or clingy polymer will strengthen the bond or connection between the mask flange 111 which externally communicates with the surface of the patient's face, i.e., the chin, nose, nose bridge cheeks, beard, etc., while increasing the physical relationship between the patient and mask flange 111, so as to provide an improved positive seal, elevating the level of comfort and fortifying the relationship between the patient's face and mask flange 111. The silicone or other plastic polymer can comprise any of a number of materials including, but in no manner limited to, adhesives, polymeric materials, plastics or PVC. The silicone or plastic polymer can be any color, shape, or size, and is not limited by the design of the mask flange 111. Further, the silicone or plastic polymer can be shaped in any pattern, texture, scheme, or arrangement of different types of polymers, materials, plastics, or PVC, as needed by a particular mask flange 111 configuration.

A passive oxygen mask 110 further comprises a gas reservoir 114, such as is shown by way of example in FIG. 1. The gas reservoir 114 is provided to at least temporarily contain an amount of oxygen, typically at a concentration of 100%, and/or other medical gases under minimal net positive pressure such that when a patient inhales while wearing the passive oxygen mask 110, an amount of oxygen and/or other medical gases may be drawn in to the patient's lungs from the gas reservoir 114. As will be appreciated, a gas reservoir 114 eliminates strain on the patient which may result from inhaling oxygen and/or other medical gases directly from a gas supply line 113. In at least one embodiment, a one way check valve is provided in a gas inlet port 112 of a passive oxygen mask 110 so as to prevent expired gas exhaled by the patient from entering the gas supply line 113 and/or the gas reservoir 114.

A passive oxygen mask 110 in accordance with the present invention further comprises an expired gas outlet port 115. Similar to gas inlet port 112, in at least one embodiment, a one way check valve may be provided in an expired gas outlet port 115 of a passive oxygen mask 110 so as to prevent the patient from inhaling air from the surrounding atmosphere or from an expired gas discharge tube 127, as is discussed in greater detail below. As will be appreciated, such an arrangement of one-way check valves in each of a gas inlet port 112 and an expired gas outlet port 115 of the passive oxygen mask 110 in accordance with at least one embodiment of the present invention will assure that the patient is only inhaling oxygen and/or other medical gases intended to be provided to the patient such as, via gas supply line 113, and further, that the expired gases exhaled by the patient exit the mask without contaminating the incoming oxygen and/or other medical gases. As a result, the respiration of the patient may be precisely monitored and controlled and/or assisted as may be needed.

Along those lines, in at least one embodiment of a passive oxygen mask and vacuum regulation system 100 in accordance with the present invention, a passive oxygen mask 110 further comprises a sampling port 116 disposed in fluid communication with an internal volume of the passive oxygen mask 110, so as to permit sampling of a gaseous mixture from the internal volume of the passive oxygen mask 110 for analysis. As shown in the illustrative embodiment of FIG. 1, a passive oxygen mask 110 further comprises the sample tube 117 disposed in fluid communication with a sampling port 116 so as to transfer an amount of a gaseous mixture from the internal volume of the passive oxygen mask 110 to one or more vapor analyzers such as may be required for monitoring a particular condition of a patient.

In at least one embodiment, an amount of a gaseous mixture sampled from the internal volume of a passive oxygen mask 110 is analyzed for end tidal carbon dioxide, or ETCO2, via capnography, wherein a capnogram is a direct monitor of the inhaled and exhaled concentrations or partial pressures of carbon dioxide, and an indirect monitor of the carbon dioxide partial pressure in the arterial blood. As is known, the difference between arterial blood and expired gas carbon dioxide partial pressures is very small in healthy individuals. Thus, monitoring ETCO2 provides a simple and non-invasive means of monitoring a patient's overall respiratory health.

In at least one further embodiment, an amount of a gaseous mixture sampled from the internal volume of a passive oxygen mask 110 is analyzed to determine the fraction of inspired oxygen, or FiO2, which is basically the amount of oxygen present in the air which is being inhaled by a patient, such as, via a passive oxygen mask 110 in accordance with the present invention. It is well known that the atmosphere of the earth comprises only about 21% by volume of oxygen, about 78% by volume of nitrogen, and about 1% of trace components such as argon, carbon dioxide, neon, helium and methane. Patients' receiving oxygen, whether via a passive oxygen mask 110 in accordance with the present invention, or via a “positive ventilation” oxygen mask as described above, are typically being provided with oxygen as a result of low blood oxygen level which may be caused any of a variety of ailments and/or conditions, and at least initially, an FiO2 level will be close to 100% in an attempt to alleviate the low blood oxygen level condition.

Looking once again to FIG. 1, a passive oxygen mask and vacuum regulation system 100 in accordance with the present invention further comprises a vacuum regulation assembly 120. As may be seen from FIG. 1, a vacuum regulation assembly 120 comprises a vacuum regulator 122 having a regulator housing 124. The regulator housing 124 in at least one embodiment includes a mask interconnect 125. A mask interconnect 125 is provided to facilitate connection of an expired gas discharge tube 127 between a passive oxygen mask 110 and a vacuum regulator 122. As may be seen from FIG. 1, an expired gas discharge tube 127 is attached in fluid communication at one end to an expired gas outlet port 115 of a passive oxygen mask 110, and at an opposite end to a mask interconnect 125 of a vacuum regulator 122.

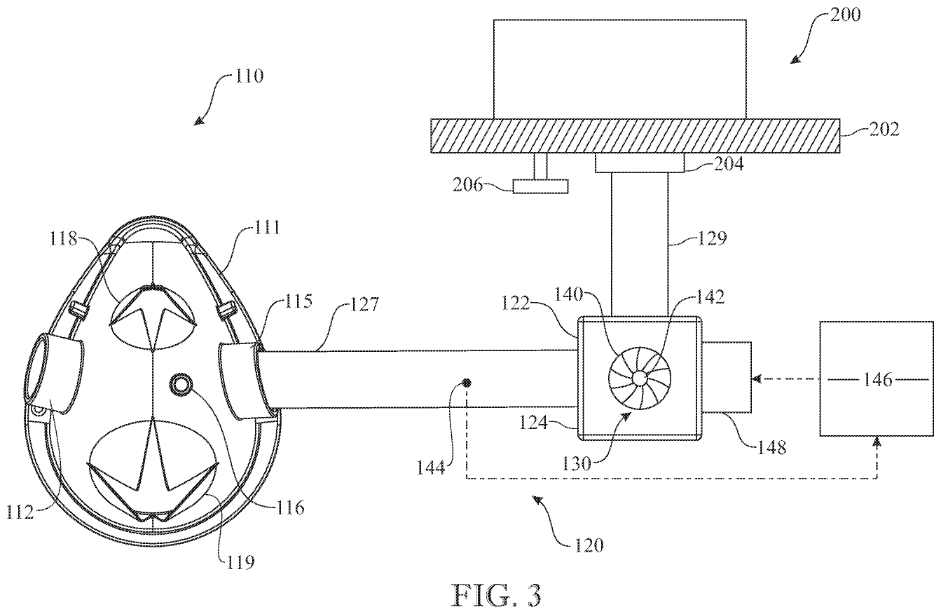

In at least one further embodiment, a vacuum regulator 122 comprises a vacuum array interconnect 128 to facilitate connection of a vacuum suction tube 129 between the vacuum regulator 122 and a vacuum array interface 202 of a vacuum scavenging array 200 such as are present in modern hospital rooms, typically behind the head of a patient bed. More in particular, a vacuum scavenging array 200 provides a central vacuum suction system which is required, often by law, to remove spent oxygen and/or other medical gases, particularly anesthetics, which are exhaled by a patient wearing an oxygen and/or other medical gas supplying mask. Typically, a vacuum scavenging array 200 provides a vacuum suction pressure in a range of about 700 millimeters of mercury negative gauge pressure to about 100 millimeters of mercury negative gauge pressure. Further, a vacuum scavenging array 200 typically includes a local vacuum controller 206, such as is shown in FIGS. 1 and 3, which allows for gross adjustment of a vacuum suction pressure at a vacuum intake 204, which are typically gross controls in increments of about 100 millimeters of mercury negative gauge pressure. With reference once again to FIG. 1, in at least one embodiment, a vacuum suction tube 129 is attached in fluid communication at one end to a vacuum array interconnect 128 of a vacuum regulator 122, and at an opposite end to a vacuum intake 204 of a vacuum scavenging array 200.

As is to be appreciated, a vacuum suction pressure of as little as 100 millimeters of mercury negative gauge pressure will adversely affect the operation of a “non-positive ventilation” oxygen mask, such as, a passive oxygen mask 110 in accordance with the present invention. Specifically, a vacuum suction pressure of 100 millimeters of mercury negative gauge pressure would essentially draw oxygen and/or other medical gases supplied to the passive oxygen mask 110 via gas supply line 113 directly out through expired gas outlet port 115 before a patient has an opportunity to inhale and process the oxygen or other medical gases, thereby defeating the purpose of the passive oxygen mask 110 itself. Furthermore, a vacuum suction pressure of as little as 100 millimeters of mercury negative gauge pressure would significantly interfere with the sampling and measurement of vital respiration parameters of a patient wearing a passive oxygen mask 110 in accordance with the present invention, for example, ETCO2 and FiO2 as described hereinabove, among others. However, once again, it is often desirable, and in some cases it is mandated, to capture the expired gases exhaled by a patient wearing a passive oxygen mask 110. This becomes particularly important when treating a patient infected with an airborne viruses and/or other airborne pathogens, such as, by way of example, a patient infected with the novel coronavirus known as COVID-19.

As such, a vacuum regulation assembly 120 in accordance with at least one embodiment of the present invention comprises a vacuum attenuation port 130 which is disposed into and through at least a portion of the regulator housing 124 of the vacuum regulator 122. More in particular, a vacuum attenuation port 130 allows a predetermined amount of ambient air proximate the vacuum regulation assembly 120 to enter the vacuum regulator 122 and to pass therethrough into a vacuum suction tube 129 interconnected to the vacuum intake 206 of the vacuum scavenging array 200, as shown throughout the figures. Thus, a vacuum attention port 130 in accordance with the present invention essentially provides an alternate source and path of airflow into the vacuum intake 206, such that the vacuum suction pressure exerted on an expired mask discharge tube 127, and thus, within an internal volume of a passive oxygen mask 100 in accordance with the present invention, can be efficiently and effectively reduced to acceptable operable levels. More in particular, the vacuum attenuation port 130 serves to attenuate the negative end expiratory pressure, or NEEP, in an expired gas discharge tube 127, and thus, at the expired gas outlet port 115 of a passive oxygen mask 110 in accordance with the present invention. As a result, the vacuum suction pressure exerted on the internal volume of the passive oxygen mask 110 is reduced to an effective yet operable level. Specifically, the vacuum attenuation port 130 of a vacuum regulation system 120 in accordance with at least one embodiment of the present invention creates a symbiotic and homeostatic relationship between an oxygen supply, an amount of removal of expired and/or waste gases, and a patient's respiratory cycle within a passive oxygen mask 110.

As will be appreciated, based upon a known vacuum suction pressure at a vacuum intake 206 of a vacuum scavenging array 200, for example, 100 millimeters of mercury negative gauge pressure, and a length and diameter of each of an expired gas discharge tube 127 and a vacuum suction tube 129, the diameter of a vacuum attenuation port 130 required to effectuate a predetermined reduction in the vacuum suction pressure present in the expired gas discharge tube 127, and thus, once again, in the internal volume of the passive oxygen mask 110 itself, can be readily determined. More particular, a diameter of a vacuum attenuation port 130 of a vacuum regulation assembly 120 may be selected such that a predetermined vacuum pressure exhibited in an expired gas discharge tube 127, and thus, at an expired gas outlet port 115 disposed in fluid communication with an internal volume of a passive oxygen mask 110, so as to safely and effectively capture and remove expired and/or waste gases from the interior of a passive oxygen mask 100, without negatively impacting a supply of oxygen and/or other medical gases to the patient via a passive oxygen mask 110 and/or without negatively impacting the sampling and analysis of an amount of a gaseous mixture from the internal volume of the passive oxygen mask 110, in accordance with the present invention.

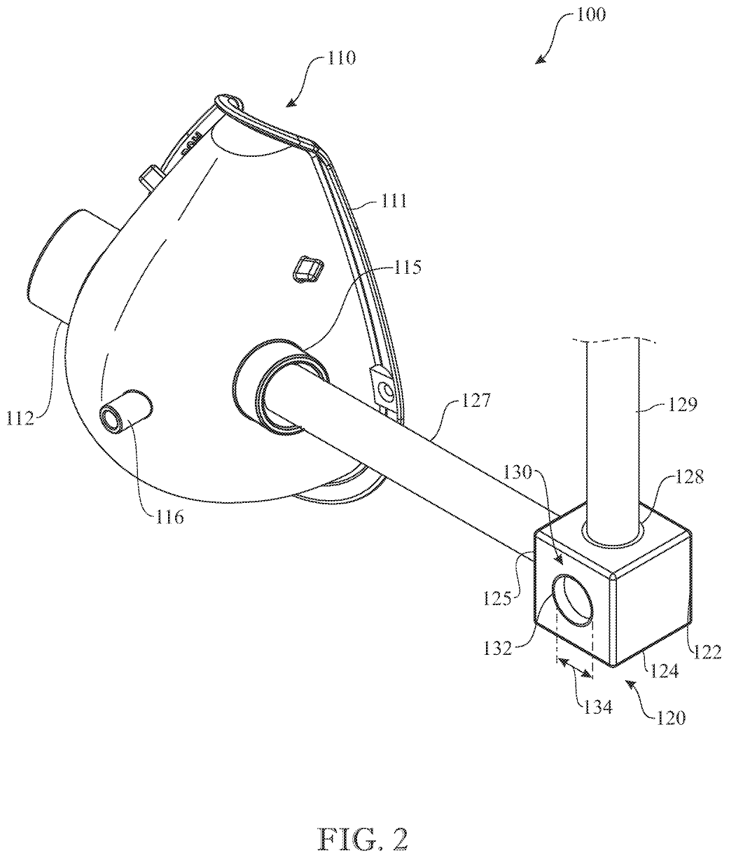

In at least one embodiment of a passive oxygen mask and vacuum reduction system 100 in accordance with the present invention, a vacuum attenuation port 130 comprises a fixed vacuum attention port 132, such as is shown by way of example in the illustrative embodiment of FIGS. 1 and 2. Further, and as may be seen best in FIG. 2, the fixed vacuum attention port 132 comprises a fixed attenuation port diameter 134. As before, based on a known vacuum suction pressure present at a vacuum intake 206 of a standard vacuum scavenging array 200, such as, by way of example, 100 millimeters of mercury negative gauge pressure, and a standardized length and diameter of both an expired gas interconnect tube 127 and a vacuum suction tube 129, a fixed attenuation port diameter 134 may be selected such that a predetermined vacuum suction pressure is exhibited in the expired gas discharge tube 127 in accordance with at least one embodiment of the present invention. In at least one embodiment, a fixed attenuation port diameter 134 of a fixed vacuum attenuation port 132 is selected such that a vacuum suction pressure exhibited in an expired gas discharge tube 127 is in a range of about 10 millimeters of mercury negative gauge pressure to about 90 millimeters of mercury negative gauge pressure. In still one further embodiment, a fixed attenuation port diameter 134 is selected such that a vacuum suction pressure exhibited in an expired gas discharge tube 127 is about 75 millimeters of mercury negative gauge pressure, and in yet one other embodiment, a fixed attenuation port diameter 134 is selected such that a vacuum suction pressure exhibited in an expired gas discharge tube 127 is about 50 millimeters of mercury negative gauge pressure. In still another embodiment, a fixed attenuation port diameter 134 of a fixed vacuum attenuation port 132 is selected such that a vacuum suction pressure exhibited in an expired gas discharge tube 127 is about 25 millimeters of mercury negative gauge pressure.

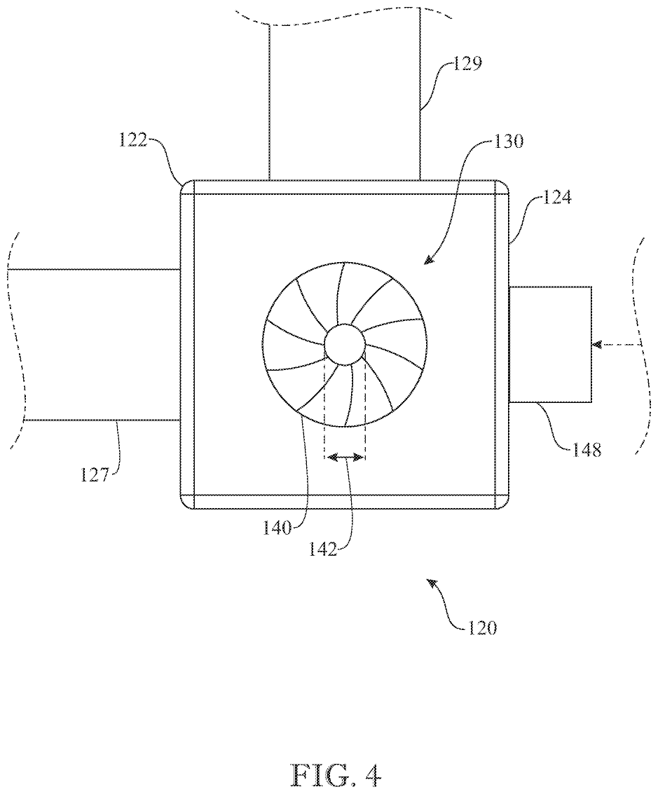

At least one alternative embodiment is contemplated in addition the embodiment shown and described hereinabove. More in particular, with reference to FIGS. 3 and 4, presented therein are a front elevation and a partial front elevation of one alternate illustrative embodiment of a passive oxygen mask and vacuum regulation system 100, in accordance with the present invention.

Looking first to FIG. 3, shown therein is an alternative illustrative embodiment of a passive oxygen mask and vacuum regulation system 100 comprising a variable vacuum attenuation port 140. As before, the system 100 includes a passive oxygen mask 110 disposed in fluid communication with a vacuum regulation assembly 120 via an expired gas discharge to 127. The passive oxygen mask 110 in one embodiment includes a nasal instrument insertion port 118 to allow one or more nasal instruments to be operatively positioned in relation to the patient through the passive oxygen mask 110. A nasal instrument insertion port 118 comprises one or more resilient flaps to engage and form a seal around the nasal instrument(s) positioned therethrough, so as to minimize release of gases through the nasal instrument insertion port 118. The passive oxygen mask 110 in at least one further embodiment includes an oral instrument insertion port 119 to allow one or more oral instruments to be operatively positioned in relation to the patient through the passive oxygen mask 110. Similar to a nasal instrument insertion port 118 in accordance with the present invention, an oral instrument insertion port 119 comprises one or more resilient flaps to engage and form a seal around the oral instrument(s) positioned therethrough, so as to minimize release of gases through the oral instrument insertion port 119.

Further, and also as before, the vacuum regulation assembly 120 is further disposed in fluid communication with a vacuum scavenging interface 202 of a vacuum scavenging array 200 such as described in detail hereinabove. More in particular, a vacuum suction tube 129 is disposed in fluid communication between the vacuum reduction assembly 120 and the vacuum intake 204 of the vacuum scavenging array 200. The vacuum regulation assembly 120 as shown in the illustrative embodiment of FIGS. 3 and 4 comprises a vacuum attenuation port 130, as before, however, the vacuum attenuation port 130 in the alternative embodiment shown in FIGS. 3 and 4 comprises a variable vacuum attenuation port 140, as stated above.

More in particular, a variable vacuum attenuation port 140 comprises a variable attenuation port diameter 142, such as is shown best in FIG. 4, and may be expanded and/or contracted so as to maintain a vacuum suction pressure exhibited in an expired gas discharge tube 127, and thus, as exhibited at an expired gas outlet port 115 disposed in communication with an internal volume of a passive oxygen mask 110 in accordance with the present invention, at one of a plurality of predetermined vacuum suction pressures. As further shown in the alternative illustrative embodiment of FIG. 3, a variable vacuum attenuation port actuator 148 is provided to affect the expansion and/or contraction of a variable vacuum attenuation port 140 as needed so as to maintain a vacuum suction pressure exhibited in an expired gas discharge tube 127 at a select one of the plurality of predetermined vacuum suction pressures.

At least one embodiment, a vacuum suction pressure sensor 144 is disposed in fluid communication with at least a portion of an expired gas discharge tube 127 so as to facilitate monitoring of a vacuum suction pressure exhibited therein. In accordance with the alternative illustrative embodiment of FIG. 3, a variable attenuation port controller 146 is provided and is disposed in communication with a vacuum suction pressure sensor 144 so as to permit continuous monitoring of a vacuum suction pressure exhibited within expired gas discharge tube 127. As further shown in FIG. 3, in at least one embodiment of the present system 100 a vacuum attenuation port controller 146 is also disposed in communication with a variable attenuation port actuator 148, wherein the variable attenuation port controller 146 is communicative with the variable attenuation port actuator 148 and instructs the actuator 148 to expand or contract the variable vacuum attenuation port diameter 142 of the variable vacuum attenuation port 140 as needed so as to maintain a vacuum suction pressure exhibited in an expired gas discharge tube 127 at a select one of a plurality of predetermined vacuum suction pressures.

It is to be appreciated that in at least one further embodiment of a passive oxygen mask and vacuum regulation system 100 in accordance with the present invention, a variable attenuation port diameter 142 of the variable vacuum attenuation port 140 may be expanded or contracted manually by an operator, once again, so as to maintain a vacuum suction pressure exhibited in an expired gas discharge tube 127 at one of a plurality of predetermined vacuum suction pressures.

In at least one embodiment, a variable attenuation port diameter 142 of a variable vacuum attenuation port 140 is selected such that a vacuum suction pressure exhibited in an expired gas discharge tube 127 is in a range of about 10 millimeters of mercury negative gauge pressure to about 90 millimeters of mercury negative gauge pressure. In still one further embodiment, a variable attenuation port diameter 142 is selected such that a vacuum suction pressure exhibited in an expired gas discharge tube 127 is about 75 millimeters of mercury negative gauge pressure, and in yet one other embodiment, a variable attenuation port diameter 142 is selected such that a vacuum suction pressure exhibited in an expired gas discharge tube 127 is about 50 millimeters of mercury negative gauge pressure. In still another embodiment, a variable attenuation port diameter 142 of a variable vacuum attenuation port 140 is selected such that a vacuum suction pressure exhibited in an expired gas discharge tube 127 is about 25 millimeters of mercury negative gauge pressure.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A passive oxygen mask and vacuum regulation system disposed in fluid communication with a vacuum scavenging array via a vacuum interface, said system comprising:
a passive oxygen mask defining an internal volume configured and dimensioned to fit over a patient's nose and mouth, said mask having a gas inlet port and an expired gas outlet port;
a vacuum regulation assembly comprising a vacuum regulator having a regulator housing;
said vacuum regulation assembly further comprising an expired gas discharge tube disposed in fluid communication between said passive oxygen mask and said vacuum regulation assembly;
said vacuum regulation assembly also including a vacuum suction tube disposed in fluid communication between said vacuum regulation assembly and the vacuum scavenging array; and
said vacuum regulation assembly further comprising a vacuum attenuation port through a portion of said regulator housing, said vacuum attenuation port at least partially defined by an attenuation port diameter dimensioned to reduce a vacuum suction pressure in said expired gas discharge tube to a predetermined vacuum suction pressure.

2. The system as recited in claim 1 wherein said passive oxygen mask further comprises a gas reservoir disposed in fluid communication with said internal volume thereof.

3. The system as recited in claim 1 wherein said passive oxygen mask further comprises a sampling port disposed in fluid communication with said internal volume thereof to permit sampling of a gaseous mixture from said internal volume of said passive oxygen mask for analysis.

4. The system as recited in claim 1 wherein said passive oxygen mask further comprises a nasal instrument insertion port.

5. The system as recited in claim 1 wherein said passive oxygen mask further comprises an oral instrument insertion port.

6. The system as recited in claim 1 wherein said vacuum regulation assembly further comprises a fixed vacuum attenuation port through a portion of said regulator housing, said fixed vacuum attenuation port at least partially defined by a fixed attenuation port diameter dimensioned to reduce said vacuum suction pressure in said expired gas discharge tube to said predetermined vacuum suction pressure.

7. The system as recited in claim 6 wherein said predetermined vacuum suction pressure is about 75 millimeters of mercury negative gauge pressure.

8. The system as recited in claim 6 wherein said predetermined vacuum suction pressure is about 50 millimeters of mercury negative gauge pressure.

9. The system as recited in claim 6 wherein said predetermined vacuum suction pressure is about 25 millimeters of mercury negative gauge pressure.

10. The system as recited in claim 1 wherein said vacuum attenuation port comprises a variable vacuum attenuation port through a portion of said regulator housing, said variable vacuum attenuation port at least partially defined by a variable attenuation port diameter dimensioned to reduce said vacuum suction pressure in said expired gas discharge tube to one of a plurality of predetermined vacuum pressures.

11. The system as recited in claim 10 wherein said vacuum regulation assembly further comprises a vacuum sensor disposed in fluid communication with said expired gas discharge line so as to allow measurement of a vacuum pressure therein.

12. The system as recited in claim 11 wherein said vacuum regulation assembly further comprises a variable attenuation port controller disposed in communication with at least said vacuum sensor disposed in said expired gas discharge tube.

13. The system as recited in claim 12 wherein said vacuum regulation assembly further comprises a variable attenuation port actuator operative with said variable vacuum attenuation port so as to vary said variable attenuation port diameter thereof.

14. The system as recited in claim 13 wherein said variable attenuation port controller is further disposed in communication with said variable attenuation port actuator, said variable attenuation port controller configured to direct said variable attenuation port actuator to expand or contact said variable attenuation port diameter so as to vary said vacuum pressure in said expired gas discharge tube as detected by said vacuum sensor to one of said plurality of predetermined vacuum pressures.

15. The system as recited in claim 14 wherein said plurality of predetermined vacuum pressures range from about 10 millimeters of mercury negative gauge pressure to about 90 millimeters of mercury negative gauge pressure.

16. The system as recited in claim 14 wherein said plurality of predetermined vacuum pressures range from about 25 millimeters of mercury negative gauge pressure to about 75 millimeters of mercury negative gauge pressure.

17. A passive oxygen mask and vacuum regulation system disposed in fluid communication with a vacuum scavenging array via a vacuum interface, said system comprising:
a passive oxygen mask defining an internal volume configured and dimensioned to fit over a patient's nose and mouth, said passive oxygen mask having a gas inlet port and an expired gas outlet port;
a vacuum regulation assembly comprising a vacuum regulator having a regulator housing, said regulator housing including a mask interconnect and a vacuum array interconnect;
said vacuum regulation assembly further comprising an expired gas discharge tube disposed in fluid communication with and interconnected between said expired gas outlet port of said passive oxygen mask and said mask interconnect of said vacuum regulation assembly;
said vacuum regulation assembly also including a vacuum suction tube disposed in fluid communication between said vacuum array interconnect of said vacuum regulation assembly and the vacuum interface of the vacuum scavenging array; and
said vacuum regulation assembly further comprising a fixed vacuum attenuation port through a portion of said regulator housing, said fixed vacuum attenuation port at least partially defined by a fixed attenuation port diameter dimensioned to reduce a vacuum pressure in said expired gas discharge tube to a predetermined vacuum pressure of less than about 100 millimeters of mercury negative gauge pressure.

18. The system as recited in claim 17 wherein said predetermined vacuum pressure is about 25 millimeters of mercury negative gauge pressure.

19. The system as recited in claim 17 wherein said predetermined vacuum pressure is about 50 millimeters of mercury negative gauge pressure.

20. A passive oxygen mask and vacuum regulation system disposed in fluid communication with a vacuum scavenging array via a vacuum interface, said system comprising:
a passive oxygen mask defining an internal volume configured and dimensioned to fit over a patient's nose and mouth, said passive oxygen mask having a gas inlet port and an expired gas outlet port;
a vacuum regulation assembly comprising a vacuum regulator having a regulator housing, said regulator housing including a mask interconnect and a vacuum array interconnect;
said vacuum regulation assembly further comprising an expired gas discharge tube disposed in fluid communication with and interconnected between said expired gas outlet port of said passive oxygen mask and said mask interconnect of said vacuum regulation assembly;
said vacuum regulation assembly also including a vacuum suction tube disposed in fluid communication between said vacuum array interconnect of said vacuum regulation assembly and the vacuum interface of the vacuum scavenging array; and
said vacuum regulation assembly further comprising a variable vacuum attenuation port through a portion of said regulator housing, said variable vacuum attenuation port at least partially defined by a variable attenuation port diameter dimensioned to reduce a vacuum pressure in said expired gas discharge tube to one of a plurality of predetermined vacuum pressures, wherein said plurality of predetermined vacuum pressures range from about 25 millimeters of mercury negative gauge pressure to about 75 millimeters of mercury negative gauge pressure.
